(19) United States
(12) Patent Application Publication
(10) Pub. No.: US 2023/0083769 A1
(43) Pub. Date: Mar. 16, 2023

(54) MACHINE LEARING BASED METHOD OF SCREENING POTENTIAL DRUG CANDIDATE, AND A METHOD THEREOF

(71) Applicant: City University of Hong Kong, Hong Kong (HK)

(72) Inventors: Chi Him Eddie MA, Hong Kong (HK); Gajendra KUMAR, Hong Kong (HK)

(21) Appl. No.: 17/883,619

(22) Filed: Aug. 9, 2022

Related U.S. Application Data
(60) Provisional application No. 63/243,749, filed on Sep. 14, 2021.

(51) Int. Cl. G16H 70/40; G06N 20/00

(52) U.S. Cl. CPC G16H 70/40; G06N 20/00

(57) ABSTRACT

A drug screening method uses electroencephalogram (EEG) or electromyogram (EMG) data applied to a ML model. EEG or EMG data is measured from a first animal species during administration of a seizure-inducing agent. A ML model is trained with the first animal species EEG or EMG data as well as measured EEG or

EMG from a second animal species such that the trained ML model is able to identify a neurological adverse event in the second animal species based on data from the first animal species. A potential drug candidate is screened by administering the potential drug candidate to the first animal species and measuring the EEG or EMG data of the first animal species during the potential drug candidate administration. The measured EEG or EMG data is applied to the ML model to determine whether there is the neurological adverse event associated with the drug candidate administration.

### Cross-Reference of Related Applications:

This present application claims the benefit of U.S. Provisional Patent Application No. 63/243,749 filed Sep. 14, 2021, which is incorporated by reference herein in its entirety.

### FIELD OF THE INVENTION

The present invention generally relates to the technical fields of pharmacology and clinical medicine. More specifically the present invention relates a machine learning (ML) based drug screening system and related method for evaluating the safety and efficacy of drug candidates on animal disease model and predict the probability in clinical study.

### BACKGROUND OF THE INVENTION

Preclinical studies are mandated by Food and Drug Administration (FDA, U.S.A.) and National Medical Products Administration (NMPA, China) before initiating first-in-human (FIH) clinical trials. It shows dose range finding and repeated dose toxicity studies crucial for investigational new drug (IND) application. It has also been reported that several drugs are rejected during the Phase I clinical trial or withdrawn from market in Phase IV clinical trial (post-marketing surveillance) due to adverse outcomes including toxicity. Overall, preclinical studies are time consuming and require huge amount of investment.

Electroencephalogram (EEG) has been used as a diagnostic tool to elucidate the pathology of neurodegenerative disease. However, it requires several hours of continuous monitoring to assess the temporal dynamics of brain activity. Basically, manual analysis of EEG signal is time-consuming and more prone to error. As a result, it is difficult to make diagnosis decision in short time for initiation of drug therapy by long-term video EEG monitoring and analysis.

Therefore, incorporation of artificial intelligence (AI) machine learning (ML) to study electroencephalogram (EEG) data appears to be an alternative to manual analysis, because it can improve selection efficiency by narrowing down the novel IND with maximum efficacy, thereby reducing the required time and investment. In addition, developing a platform of screening IND acting on central nervous system (CNS) during the toxicological study and evaluating the adverse effects and therapeutic index also appears to be a promising tool to assist and even substitute some preclinical stage of studies. Practically, the FDA requires these toxicological studies in a minimum of two animal species before FIH dosing, where large number of animals are involved, leading to increased cost and time for drug development and entry of phase I clinical trial. Furthermore, a ML model can be trained to predict the outcome of human disease condition based on a small animal disease model such as rodent.

Generally, conventional drug screening process takes 2 weeks to 6 months, and the cost of it is huge. In other words, the screening process for a drug candidate consumes considerable resources. And after the screening, the outcomes of safety clinical tests for the drug candidate remain unknown. It is a big risk for not only the investors and pharmaceutical industries but also the volunteer subjects of clinical trials. These problems highlight the need of a drug candidate screening platform that possesses the ability to screen drug candidates in a high accuracy and efficient way and even has a preliminary safety evaluation effect.

Therefore, the present invention provides a reliable, efficient and reproducible method and system for screening potential drug candidates by assessing brain activities of subjects administered with drug candidates with a combination of ML techniques.

### SUMMARY OF THE INVENTION

It is an objective of the present invention to provide a ML based method of screening a potential drug candidate to treat a disease and/or condition based on assessment of electroencephalogram (EEG) or electromyogram (EMG), and a system thereof.

In accordance with a first aspect of the present invention, the present invention provides a ML based method of screening a potential drug candidate.

The method comprises measuring EEG or EMG data of a first animal species during administration with a seizure-inducing agent. The EEG or EMG data is processed and segmented equally into a set of time sliding window EEG or EMG data by a data acquisition unit.

A seizure related signal is extracted from the set of time sliding window EEG and EMG data by a feature extraction unit and processed into a seizure related spectrogram or periodogram by a classification unit, wherein the classification unit saves and annotate the seizure related spectrogram or periodogram as normal phase or seizure phase.

The method further includes training a ML model with the first animal species EEG or EMG data as well as measured EEG or EMG from a second animal species such that the trained ML model is able to identify a neurological adverse event or to determine treatment efficacy.

In a further aspect, the training includes pre-processing the EEG or EMG signals, extracting seizure related signals from the EEG or EMG signals from pre-processed signals, converting the extracted signals into two-dimensional images for classification and annotation, classifying the two-dimensional images into seizure and normal events and using a ML model to create a plurality of feature maps followed by generating a plurality of feature vectors from the plurality of feature maps by a plurality of fully connected layers.

In one aspect, the ML model is trained to evaluate the change in power of frequency, polyspike sharp wave, or amplitude of EGG or EMG.

The method further includes screening the potential drug candidate by administering the potential drug candidate to the first animal species and measuring the EEG or EMG data of the first animal species during the potential drug candidate administration.

The method applies the measured EEG or EMG data taken during the potential drug candidate administration to the ML model to determine whether there is the neurological adverse event associated with drug administration.

The first animal species may be a rodent while the second animal species may be human.

In one aspect, the neurological adverse event comprises seizure, or other aspects of epilepsy.

### BRIEF DESCRIPTION OF THE DRAWINGS

Embodiments of the invention are described in more details hereinafter with reference to the drawings, in which:

FIG. 1 illustrates an EEG feature extraction process and testing of artificial intelligence (AI) model according to an embodiment of the present invention;

FIG. 2 schematically depicts a convolutional neural network (CNN) of AI model according to an embodiment of the present;

FIG. 3 schematically depicts how to assess the translational potential of pre-clinical studies to clinical application (bench to bed) according to an embodiment of the present invention;

FIG. 4 is a schematic diagram showing electrode distribution in acquisition of electroencephalogram (EEG) and electromyogram (EMG) in mice; and

FIG. 5 depicts schematically depicts a mouse model of epilepsy and EEG recording paradigm of the present invention.

### DETAILED DESCRIPTION

In the following description, methods of drug screening and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The invention includes all such variation and modifications. The invention also includes all of the steps and features referred to or indicated in the specification, individually or collectively, and any and all combinations or any two or more of the steps or features. Other aspects and advantages of the invention will be apparent to those skilled in the art from a review of the ensuing description

In accordance with a first aspect, the present invention provides a method of screening potential candidates of an active ingredient to treat a disease and/or condition based on assessment of electroencephalogram (EEG) and electromyogram (EMG). In order to develop the drug screening method, an artificial intelligence (AI) machine learning (ML) based EEG platform is constructed. The ML model is trained with EEG data from animals undergoing induced seizures. Using this EEG data, along with EEG data from humans experiencing seizures, trains the ML model to correlate the animal model seizure data to human seizures. Using the trained ML model, drugs to be screened can be administered to an animal and the measured EEGs from these animals can be used to predict whether a seizure or other adverse neurological events may occur from the drug being screened. Alternatively, using the EEGs taken during drug administration, the efficacy of the drug to treat seizures or other neurological symptoms can be evaluated using the trained ML model.

In order to generate the EEG data for training the ML model, a seizure inducing drug is administered to rodents. The rodents are chosen for setting up and training the platform. EEGs are performed on mice who are induced into having epileptic seizures through administration of a suitable medication, such as kainic acid. EEG or EMG raw data is obtained from central nervous system and associated tissue(s) therewith through one or more electrodes implanted or inserted into one or more corresponding body parts of the subject and sending the obtained EEG or EMG raw data to a data acquisition unit.

The EEG data is pre-processed for removing artifacts, interpolating bad channels. The data is filtered for the best quality by using a notch filter of 60 Hz and a high pass filter of 0.5 Hz. Initially, all of the signals are filtered in a defined frequency range (1-100 Hz) and time domain. The signals are segmented equally and nonoverlapping for a slide window analysis. EEG signals with high amplitudes (>1,000 μV) are discarded to stabilize feature generation and remove the outlier values. A sliding window with time analysis is an important factor to split the raw

EEG data into segments for feature extraction. Due to the non-stationary nature of EEG signals, a time window sliding ensures the stability of data while overlapping sliding window and the continuity of data. Based on the pre-processing result, the sliding time window for the normal and seizure data is set at 2 seconds.

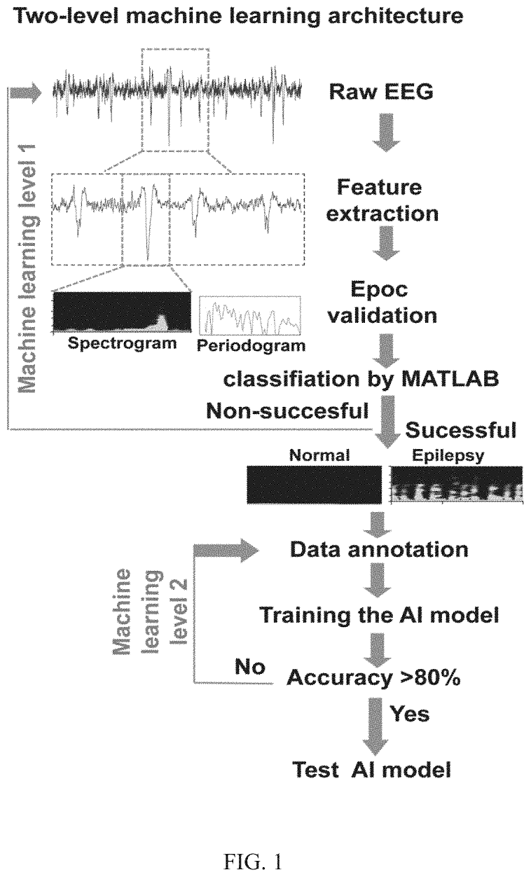

As shown in FIG. 1, a two-level ML method is conducted for the EEG feature extractions. The original EEG signal, spectrogram and periodogram are tested first, based on epoch. A spectrogram with a 2 second time interval classifies the EEG data into normal (pre-ictal) and epileptic (ictal) portions, and annotation is further performed. A ML model is trained by the following procedure: if accuracy is >80%, then it is processed for testing; otherwise, go back to annotate the data again.

In a preferred embodiment, various parameters of EEG data related to a seizure event of epilepsy are extracted. A spectrogram, absolute power of the frequency spectrum, and the original EEG signal after pre-processing into 2 second segments are generated. After that, the spectrogram signal is validated as per time, and data is exported as an image file (under e.g., the JPEG format) by using MATLAB. Furthermore, all the spectrogram images are annotated as pre-ictal (no seizure) or ictal (seizure) events. An ictal phase is characterized by an increased power of frequency in the defined time (2 seconds) and validated with the EEG signal as an increased polyspike sharp wave (20-70 milliseconds) and increased amplitude. Overall, 9,200 annotated data are generated and collected (including 5,000 annotated as normal and 4,200 annotated as epilepsy) from the epileptic mouse model.

In addition to the annotated data of mice, an additional 9,000 (5,000 normal and 4,000 epilepsy) EEG spectra were collected from epileptic patients. In brief, the EEG data of epileptic patients is collected from Shenzhen Children's Hospital, China. Electrodes are positioned as per the international 10-20 system, and the EEG data are sampled at 1,000 Hz. Seizure events are labelled by board certified epilepsy clinical experts based on the EEG signal characteristics such as polyspikes discharge, slow spike-wave activity and high amplitude sharp waves, and all the human EEG data are processed by the above procedure to generate annotated data.

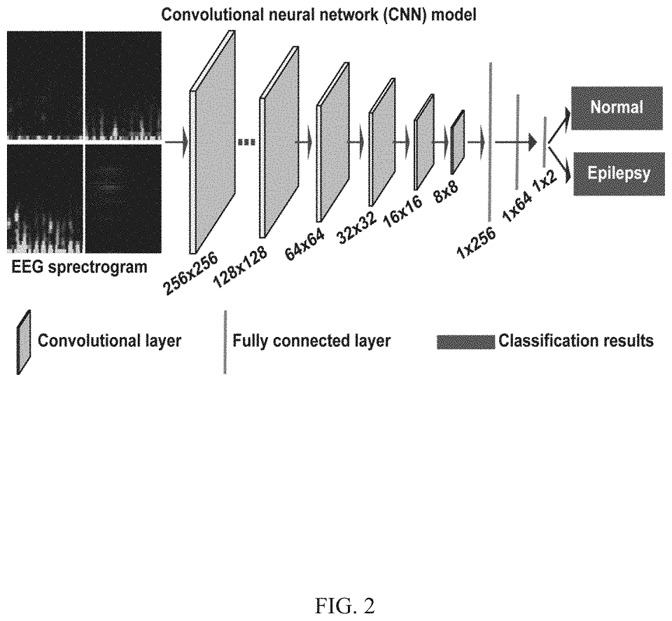

Next, all the annotated data is used for training and testing the ML model, which may be implemented by a convolutional neural network (CNN). Referring to FIG. 2, shown is the convolutional neural network (CNN) model used in the present invention, which includes six convolutional layers and two fully connected layers. Every convolutional layer contains a convolution operation such as a batch normalization operation, non-linear activation operation, and max-pooling operation. In FIG. 2, each vertical plane at each of the convolutional layers represents the relative size of the feature map after the operation of each of the convolutional layers.

After the operations by six convolutional layers, the spectrogram image is encoded into 8×8×128 feature map following with a global L2 pooling operation compresses the feature map into a 1×128 feature vector. The last 2 connected layers calculate the probability of normal or epilepsy phase in EEG data.

In one embodiment, the CNN model serves as an autoencoder in the present invention, where the convolutional layers and fully connected layers encode the spectrogram (power change in frequency with time) into a (i.e., 1×128 dimensional) feature vector. Thereafter, the last fully connected layers predict the probability of ictal or pre-ictal stage of epilepsy. Every convolutional layer contains a convolution operation, a batch normalization operation, a non-linear activation operation, and a max-pooling operation. After the operations by six convolutional layers, the spectrogram image is encoded into an 8×8×128 feature map, then a global L2 pooling operation is used to compress the feature map into a 1×128 feature vector. The last two connected layers calculate the probability of a normal phase or epilepsy phase in EEG recording. Image of each time point is resized to 256×256 for neural network.

In one test, 5,000 mice annotated data are randomly selected as for a training data set; 3,000 data are selected for validation; and 2,000 for testing. On the other hand, all of the human data is used as testing samples to compare the differences between human and mouse EEG signals. In order to validate the translational application of the ML model from pre-clinical to clinical, mouse data sets are used to train and debug the CNN model, and human data sets are used to test the effect of the model.

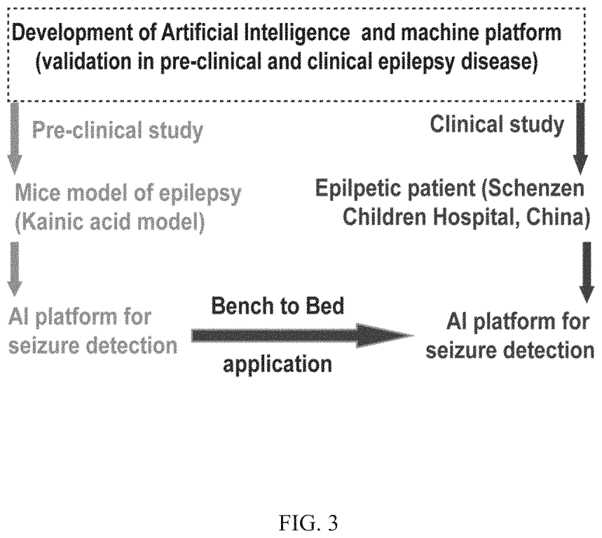

Referring to FIG. 3 for the following description. A ML platform is developed for the pre-clinical (Kainic acid-induced epilepsy) study and the seizure detection efficiency is evaluated. Furthermore, clinical (epileptic patients) EEG data are analyzed using the pre-clinical AI platform to demonstrate the bench-to-bed application of the trained ML model.

Once the ML model is trained, the trained ML model can predict adverse drug outcomes in human when new drugs are administered to rodent. By correlating rodent EEG data with human EEG data, the trained model can determine, based on the new EEG data from the new drug, whether that drug would cause an adverse outcome in humans without the need of testing the new drug on human volunteers.

Thus, the use of the trained ML model involves selecting a drug to be screened. The potential drug candidate is administered to an animal of the same species as that used to train the ML model. During the drug candidate's administration, EEG or EMG data is measured for that animal. The measured EEG or EMG data taken during the drug candidate administration is applied to the ML model to determine whether there is the neurological adverse event associated with drug administration. Alternatively, the drug may be administered to a rodent with induced seizures to determine whether the drug has efficacy in treating seizures.

### EXAMPLE 1

EEGs are performed on mice having induced epileptic seizures through the administration of a seizure-inducing agent.

All animal experiments are conducted in accordance with the guideline of Institutional Animal Care and Use Committee (IACUC), City University of Hong

Kong and approved by Department of Health, HKSAR.

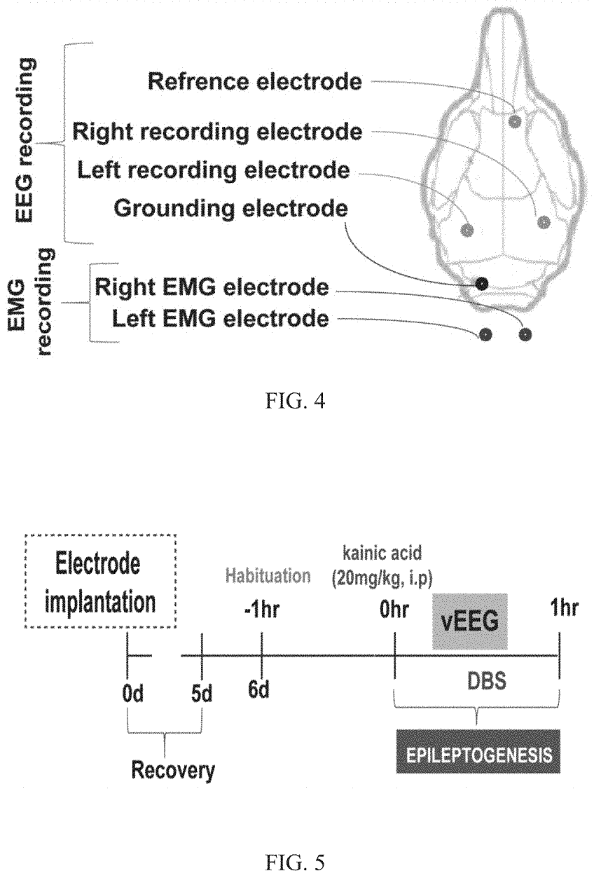

Adult C57BL/6 male mice (8-12 weeks old) are used in all the experiments. Mice are anesthetized with ketamine/xylazine and placed in stereotactic frame (Stoelting, U.S.A.). A midline skin incision (10 mm) is made to expose the cranium and epidural EEG screw electrode is fixed over parietal cortex, reference over cerebellum and grounding on frontal bone (coordinates from reference point, Bregma: right temporal cortex: anteroposterior (AP) —2.3 mm, mediolateral (ML) +1.5 mm, Dorsoventral (DV) −0.2 mm form skull; left temporal cortex: AP −2.3 mm, ML −1.5 mm, DV −0.2 mm); reference electrode: AP +2.0 mm, ML +2.0 mm, DV −0.2 mm; ground electrode: AP −6.7 mm, ML −2.0 mm, DV −0.2 mm). Two EMG electrodes are inserted into a nuchal muscle (neck) to check the movement. As shown in FIG. 4, EEG electrodes are implanted in left and right temporal cortex. EMG electrodes are inserted in left and right trapezius muscle. Reference electrode over frontal bone and grounding electrode over cerebellum are used in common for EEG and EMG.

As shown in FIG. 5, the mice are habituated in the recording chamber before Kainic acid (KA) injection (20 mg/kg, i.p) after 5 days of recovery. The dosage of kainic acid (KA, Sigma, U.S.A) is optimised for inducing seizure in mice, where 20 mg/kg (i.p.) is considered suitable for inducing seizures without high levels of mortality. EEG electrode implanted mice are connected with a data acquisition system (Medusa, Bio-Signal Technologies, U.S.A.) with video recording set up in soundproof box and have access to food and water for 24 hrs. EEG are sampled at 1,000 Hz using high pass (0.3 Hz) and low pass (100 Hz) filter. Mice are habituated for 1 hr followed by 1 hr of baseline recording. Also, EEG-video (vEEG) is recorded immediately after KA injection for 1 hr. And seizure symptoms of epilepsy are evaluated by using video recording correlated with corresponding EEG activity.

The seizure severity is scored as per modified Rascin's scale. Rascin's scale is a seven-point scoring system: Stage 0, whiskers trembling; Stage 1, sudden behavioural arrest; Stage 2, facial jerking; Stage 3, neck jerks; Stage 4, clonic seizure (sitting); Stage 5, clonic, tonic—clonic seizure (lying on belly); Stage 6, clonic, tonic-clonic seizure (lying on side) and wild jumping and Stage 7, tonic extension, possibly leading to respiratory arrest and death. Seizure score, latency to myoclonus, clonus, generalised tonic-clonic (GTC), duration of clonus and GTC are assessed for each mouse. According to Rascin's scale, mice with 4-5 stage are considered as epileptic. Mice are excluded from the study if behavioural response is either sub-convulsive or fatal.

### EXAMPLE 2

The mice and human annotated data are screened by a known AI model (VGG Model, by Oxford, the U.K.) to compare the efficacy of the present invention. The comparative result is shown in Table 1.

As shown in Table 1, the comparative results suggest that the present model achieves the accuracy of 95.3% in mouse EEG and 95.1% in human EEG, whereas Oxford model only shows an accuracy of 93.7% in mice and 92.8% in human EEG. Next, the translational capability of the present model is tested by training with mouse data followed with test by human data. Surprisingly, the present model achieves high accuracy (82.6%) compared with the Oxford model (76.2%).

TABLE 1

| Model | Training | Test | Accuracy |
|---|---|---|---|
| Present Model | Mouse | Mouse | 95.3% |
| | Human | Human | 95.1% |
| | Mouse | Human | 82.6% |
| VGG Model (Prior Art) | Mouse | Mouse | 93.7% |
| | Human | Human | 92.8% |
| | Mouse | Human | 76.2% |

The mouse model of epilepsy and epileptic patient data demonstrates that the accuracy of the present model is over 90%. Further, training loss dynamics, accuracy dynamics and receiver operating characteristics (ROC) show equal performance in mice and human EEG data, suggesting that the present model is also stable. Generally speaking, the present ML platform is versatile and universal in nature. Therefore, the results suggest that the present platform may be applied to screen the safety and efficacy of drugs in a wide range of neurological disorders.

### INDUSTRIAL APPLICABILITY

The present method incorporates a reliable and reproducible diagnosis technique for automated detection and prediction of disease using machine learning such as convolutional neural network (CNN). Mouse models of epilepsy and epileptic patient's data are used in some preferred embodiments. In those embodiments, training and accuracy of AI model shows over 90% efficiency. Further, the training loss dynamics, accuracy dynamics and receiver operating characteristics (ROC) showed equal performance in mice and human EEG data, suggest stability of the present AI model. The present AI platform is also versatile and universal in nature. Therefore, the present AI platform has potential to screen the safety and efficacy of drugs in a wide range of neurological disorders such as epilepsy, Alzheimer's and Parkinson's disease, anxiety and depression, obsessive-compulsive disorder, stroke, movement disorder (ataxia) and neurotoxicological study (alteration of brain signals due drugs or chemical exposure).

The logical functional units, modules, processors, and pre-processors of the prediction and knowledge transfer ML and DL models in accordance with the embodiments disclosed herein may be implemented using computing devices, computer processors, or electronic circuitries including but not limited to application specific integrated circuits (ASIC), field programmable gate arrays (FPGA), and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

All or portions of the methods in accordance to the embodiments may be executed in one or more computing devices including server computers, personal computers, laptop computers, mobile computing devices such as smartphones and tablet computers.

The embodiments include computer storage media, transient and non-transient memory devices having computer instructions or software codes stored therein which can be used to program computers or microprocessors to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A drug screening method using electroencephalogram (EEG) or electromyogram (EMG) data applied to a ML model, comprising:
measuring EEG or EMG data of a first animal species during administration of a seizure-inducing agent;
training the ML model with the first animal species EEG or EMG data as well as measured EEG or EMG from a second animal species such that the trained ML model is able to identify a neurological adverse event or treatment efficacy in the second animal species based on data from the first animal species;
screening a potential drug candidate by administering the potential drug candidate to the first animal species and measuring the EEG or EMG data of the first animal species during the potential drug candidate administration; and
applying the measured EEG or EMG data taken during the potential drug candidate administration to the ML model to determine whether there is the neurological adverse event associated with the drug candidate administration.

2. The method of claim 1, wherein the ML model is trained to evaluate the change in power of frequency, polyspike sharp wave or amplitude of EGG or EMG.

3. The method of claim 1, wherein the first animal species is rodent.

4. The method of claim 1, wherein the second animal species is human.

5. The method of claim 1, wherein the neurological adverse event comprises a seizure.

6. The method of claim 1, wherein the training comprises:
pre-processing the EEG or EMG signals, extracting seizure related signals from the EEG or EMG signals from pre-processed signals;
converting the extracted signals into two-dimensional images for classification and annotation;
classifying the two-dimensional images into seizure and normal events; and
using an autoencoder based on a convolutional neural network (CNN) to create a plurality of feature maps followed by generating a plurality of feature vectors from the plurality of feature maps by a plurality of fully connected layers.

7. A machine learning (ML) based system for screening a potential drug candidate based on electroencephalogram (EEG) or electromyogram (EMG), comprising:
a plurality of electrodes configured for recording EEG or EMG signals;
a data acquisition unit configured for receiving the EEG or EMG signals, wherein the data acquisition unit comprises a pre-processor;
a feature extraction unit configured for extracting seizure related signals from the EEG or EMG signals output by the data acquisition unit and converting the extracted signals into two-dimensional images for classification and annotation;
a classification unit configured for classifying the two-dimensional images from the feature extraction unit into seizure and normal events and annotating thereof according to the classification result; and
an autoencoder based on a convolutional neural network (CNN) for encoding the classified and annotated images output by the classification unit into a plurality of feature maps in different dimensions according to a sequence of convolutional layers followed by generating a plurality of feature vectors from the plurality of feature maps by a plurality of fully connected layers arranged subsequent to the plurality of convolutional layers.

8. The system of claim 7, wherein the pre-processor removes high amplitude signals and outlier value from the EEG or EMG signals and segments the pre-processed signals equally according to a time sliding window, followed by recombining the segmented signals with overlapping sliding window characteristic.

9. The system of claim 8, wherein the pre-processor comprises at least a low pass and high pass filters arranged in sequence.

10. The system of claim 8, wherein the time sliding window is approximately 2 seconds.

11. The system of claim 7, wherein the seizure related signals comprise increase in power of frequency, polyspike sharp wave or amplitude.

12. The system of claim 7, wherein the two-dimensional images comprise spectrogram and periodogram.

13. The system of claim 7, wherein the classification unit comprises MATLAB to store and annotate the two-dimensional images.

14. The system of claim 7, wherein the CNN comprises six convolutional layers and two fully connected layers arranged in sequence to resize the annotated image data into the plurality of feature maps sequentially by the six convolutional layers in a descending order of dimensions from 256×256, 128×128, 64×64, 32×32, 16×16 and 8×8, respectively, followed by generating the feature vectors by the two fully connected layers from 1×256, 1×64 and finally outputting an 1×2 feature vector for prediction of probability of the seizure incidence arising from and/or associated with the administration of the potential drug candidate to said subject.
